(19) United States
(12) Patent Application Publication
(10) Pub. No.: US 2024/0399069 A1
(43) Pub. Date: Dec. 5, 2024

(54) SAFETY NEEDLE FOR ADMINISTERING MEDICATION AND WITHDRAWING BIOLOGICAL FLUIDS

(71) Applicant: KING SAUD UNIVERSITY, Riyadh (SA)

(72) Inventors: Mohd Aftab ALAM, Riyadh (SA); Fahad Ibrahim AL-JENOOBI, Riyadh (SA)

(21) Appl. No.: 18/204,557

(22) Filed: Jun. 1, 2023

(51) Int. Cl. A61M 5/32; A61B 5/15

(52) U.S. Cl. CPC A61M 5/32; A61B 5/150389

(57) ABSTRACT

A safety needle for administering medication and withdrawing biological fluids includes a hollow needle tube including a proximate end, an opposing distal end, and a lumen extending along the length of the shaft from the proximate end to the distal end. The shaft includes an adapter at the proximate end and a beveled tip the distal end. The adapter is configured for connecting the needle to a syringe. An optional collar tube may be mounted on the shaft to limit insertion of the needle into the body. A plurality of visible depth indication bands or rings can be provided on the shaft. The bands can have various colors or various shades of one color. The bands can have uniform or non-uniform widths, with each band being a different distance from the beveled tip.

### BACKGROUND

### 1. Field

The disclosure of the present patent application relates to hypodermic needles, and particularly, to a safety needle for accurate medication administration and bodily fluid withdrawal.

### 2. Description of the Related Art

Hypodermic needles are used in veterinary and human hospitals and clinics in combination with syringes or cannulas for injecting medications into or withdrawing fluid from human or animal bodies. While injecting medication or withdrawing bodily fluid, it is important for the practitioner to be aware of how deep to insert the needle and the proper angle of insertion. An incorrect angle or depth of needle insertion may cause injury. Insufficient insertion of the needle may lead to a failed injection or leakage of fluid out of the body.

Thus, a safety needle for administering medication and withdrawing biological fluids solving the aforementioned problems is desired.

### SUMMARY

A safety needle for administering medication and withdrawing biological fluids includes a hollow needle tube or shaft including a proximate end, an opposing distal end, and a lumen extending along the length of the shaft from the proximate end to the distal end. The shaft includes an adapter at the proximate end and a beveled tip the distal end. The adapter is configured for connecting the needle to a syringe. In an embodiment, the adapter is tapered and includes a first end and a second end that is larger in diameter than the first end. An optional collar tube may be mounted on the shaft to limit insertion of the needle into the body. A plurality of visible depth indication bands or rings can be provided on the shaft. The depth indication bands may include generally circular indicia that extend along the full circumference of the shaft or half of the circumference of the shaft. The bands can have various colors or various shades of one color. The bands can have uniform or non-uniform widths, with each band being a different distance from the beveled tip.

One or more safety needles may be provided in a pre-packaged kit. The kit may include a separate guide or a guide printed on the exterior surface of the package. The guide provides the distance from the bevel tip to the closest edge of each depth identification band. The guide may include a picture of the safety needle indicating the depth of each band.

These and other features of the present subject matter will become readily apparent upon further review of the following specification.

### BRIEF DESCRIPTION OF THE DRAWINGS

FIG. 1 is an environmental perspective view of a safety needle for administering medication and withdrawing biological fluids mounted on a syringe.

FIG. 2 is an enlarged side view of an embodiment of the safety needle for administering medication and withdrawing biological fluids of FIG. 1, showing the needle mounted on the syringe.

FIG. 3 is a side view of an embodiment of the safety needle for administering medication and withdrawing biological fluids of FIG. 1, showing the details thereof.

FIG. 4 is a side view of an embodiment of the safety needle for administering medication and withdrawing biological fluids of FIG. 1, showing the details thereof.

FIG. 5 is a guide for use with the safety needle for administering medication and withdrawing biological fluids of FIG. 1.

Similar reference characters denote corresponding features consistently throughout the attached drawings.

### DETAILED DESCRIPTION OF THE PREFERRED EMBODIMENTS

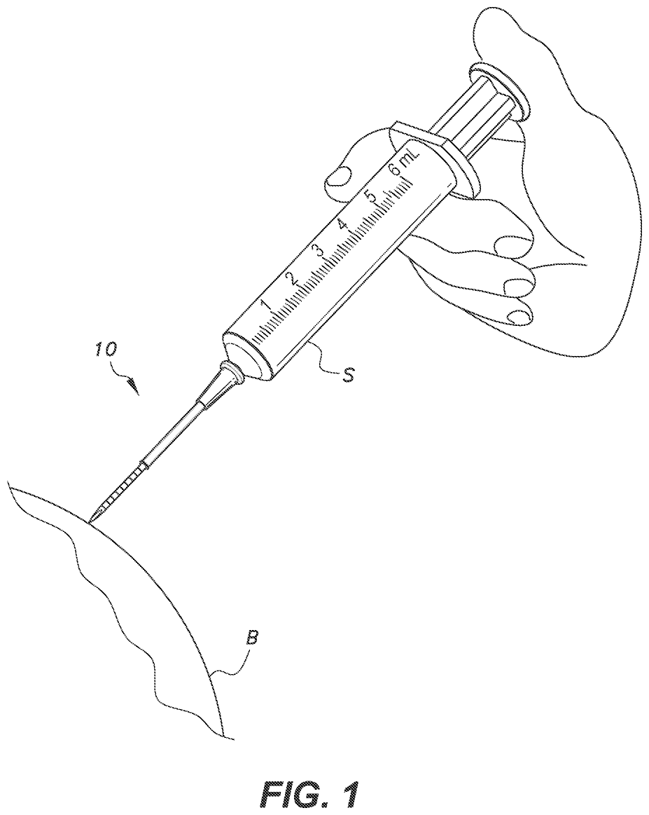

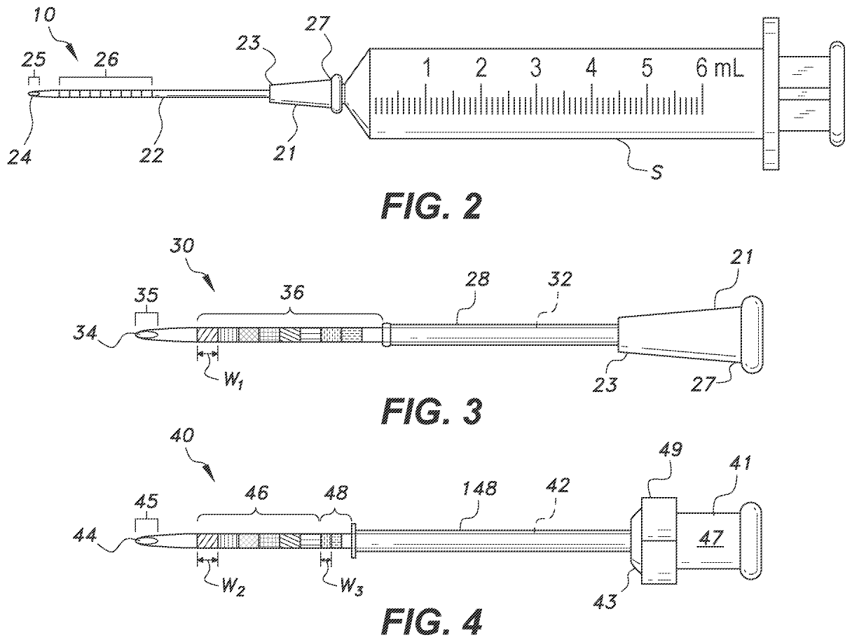

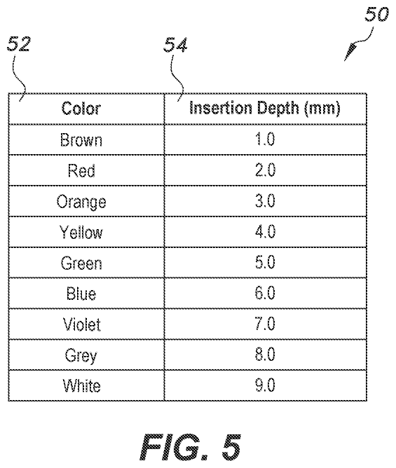

Referring to FIG. 1, the safety needle for administering medication and withdrawing biological fluids 10 is shown attached to a syringe S. The details of the safety needle 10 are shown in FIG. 2. The safety needle 10 includes a hollow needle tube or shaft 22 including a proximate end, an opposing distal end, and a lumen 24 extending along the length of the shaft from the proximate end to the distal end. The shaft 22 includes an adapter 21 at the proximate end and a beveled tip 25 at the distal end. The adapter 21 is configured for connecting the needle 22 to a syringe S or cannula. In an embodiment, the adapter 21 is tapered and includes a first end 23 and a second end 27 that is larger in diameter than the first end 23. An optional collar tube 28 may be mounted on the shaft 22 to limit insertion of the needle 10 into the body B. The collar tube 28 can be adjacent to the adapter. A plurality of visible depth indication bands or rings 26 can be provided on the shaft 22, e.g., proximate the distal end of the shaft 22. The depth indication bands 26 may include generally circular indicia that extend along the full circumference of the shaft 22 or half of the circumference of the shaft 22. The bands 26 can have various colors or various shades of one color. Each of the bands 26 can represent a different distance from the beveled tip 25. In an embodiment, the bands 26 are half-circumferential and on the same side of the needle shaft as the bevel 25 to allow a user to see the bands 26 while inserting the needle 10 into the body B. In an embodiment, a first end of the collar tube 28 can contact the adapter 21 and a second end of the collar tube 28 can be aligned with one of the bands 26 to limit insertion of the needle beyond the second end of the collar tube 28.

In an embodiment, the bands 26 are different shades of grey (greyscale), or different shades of any other color to distinguish the bands 26 from each other. The bands 26 are formed on the needle shaft 22 and are spaced from the bevel tip at pre-selected distances. The bands 26 may all have the same width or the bands may have non-uniform widths.

Referring to FIG. 3, a further embodiment of the safety needle 30 is shown. As with the previous embodiment, the safety needle 30 includes a hollow needle tube or shaft 32 with an adapter 21 at a first end, a beveled tip 35 at a second end, and a lumen 34 extending from the first end to the second end. The adapter 21 is tapered and includes a first end 23 and a second end 27 that is larger than the first end 23. An optional collar tube 28 may be mounted on the shaft 32, to limit insertion of the needle 10 into the body B. The safety needle 30 also includes visible depth indication color bands 36 on its surface at different distances from the needle bevel 35. Each of the bands 36 are a different color or shade and have the same uniform width W₁. For example, the bands 26 may have a width ranging from about 0.5 mm to about 5 mm or a width ranging from about 1 mm to about 2 mm. In an embodiment, a first end of the collar tube 28 can contact the adapter 21 and a second end of the collar tube 28 can be aligned with one of the bands 36 to limit insertion of the needle beyond the second end of the collar tube 28.

A further embodiment of the safety needle, designated 40, is shown in FIG. 4. As with the previous embodiments, the safety needle 40 includes a hollow needle tube or shaft 42 with an adapter 41 at a first end, a beveled tip 45 at a second end, and a lumen 44 extending from the first end to the second end. The adapter 41 differs from the adapters of the previous embodiments. The adapter 41 includes a tapered section 43 attached to the shaft 42, a cylindrical section 47 attachable to a syringe S or cannula, and a hexagonal section 49 between the tapered section 43 and the cylindrical section 47. An optional collar tube 148 may be mounted on the shaft to limit the insertion of the needle 40 into the body B. The safety needle 40 also includes lower and upper visible depth indication color bands 46, 48, respectively. The bands 46, 48 can have different widths. Each of the bands 46, 48 can have a different color or shade. The width W₂ of the lower bands 46 (the bands nearest to the bevel) can be selected based on the application of the needle (needle depth). For example, the lower bands 46 can have a width ranging from about 2 mm to about 5 mm. The upper bands 48 can have a width W₃ ranging from about 1 mm to about 2 mm for more accurate depth measurement. In an embodiment, a first end of the collar tube 148 can contact the adapter 41 and a second end of the collar tube 148 can be aligned with one of the bands 46, 48 to limit insertion of the needle beyond the second end of the collar tube 148.

A kit can include one or more safety needles 10, 30, 40 shown in FIGS. 1-4 and an explanatory guide 50. The guide 50 may be a separate item included within the package interior or printed on the exterior surface of the exterior kit packaging. The guide 50 can include a listing of the color or shade of each band 52 and a listing of insertion depth measurements 54 represented by each band. The insertion depth is the distance (depth) from the bevel tip to the edge of each depth identification band closest to the tip. The guide 50 may also include a picture of the safety needle such as shown in FIGS. 3-4 with a numerical indication of the insertion depth corresponding to each band.

It is to be understood that the safety needle for administering medication and withdrawing biological fluids is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A safety needle for administering medication and withdrawing biological fluids, comprising:
a needle shaft having a first end, a second end and a lumen extending from the first end to the second end, the second end including a beveled tip;
an adapter attached to the first end of the needle shaft;
a collar tube mounted on the needle shaft, having a first end and a second end, configured to limit an insertion depth of the safety needle into a body; and
a plurality of depth indication bands extending at least partially along a circumference of the needle shaft, each band of the plurality of depth indication bands having a width and being positioned at a different distance from the beveled tip,
wherein a width of one of the plurality of depth indication bands is greater than a width of a remainder of the plurality of depth indication bands,
wherein a width of the one of the plurality of depth indication bands nearest to the beveled tip is angled with respect to the first end of the collar tube thereby providing an indication of a proper angle of insertion of the needle shaft, and
wherein the first end of the collar tube contacts the adapter and the second end of the collar tube aligns with one of the plurality of depth indication bands.

2. The safety needle for administering medication and withdrawing biological fluids as recited claim 1, wherein each of the plurality of depth indication bands is a different color.

3. The safety needle for administering medication and withdrawing biological fluids as recited in claim 1, wherein each of the plurality of depth indication bands is a different shade of the same color.

4. The safety needle for administering medication and withdrawing biological fluids as recited in claim 3, wherein each of the plurality of depth indication bands is a different shade of grey.

5. (canceled)

6. The safety needle for administering medication and withdrawing biological fluids as recited in claim 1, wherein the adapter comprises:
a small diameter end attached to the first end of the shaft; and
a large diameter end attachable to a syringe or cannula, wherein the adapter is tapered from the small diameter end to the large diameter end.

7. (canceled)

8. The safety needle for administering medication and withdrawing biological fluids as recited in claim 1, wherein the adapter comprises:
a tapered section attached to the first end of the shaft;
a cylindrical section attachable to a syringe or cannula; and
a hexagonal section between the tapered section and the cylindrical section.

9-10. (canceled)

11. A kit for administering medication and withdrawing biological fluids comprising at least one safety needle, the at least one safety needle, comprising:
a needle shaft having a first end, a second end and a lumen extending from the first end to the second end, the second end including a beveled tip;
an adapter attached to the first end of the needle shaft;
a collar tube mounted on the needle shaft, having a first end and a second end, configured to limit an insertion depth of the safety needle into a body; and
a plurality of depth indication bands extending at least partially along a circumference of the needle shaft, each band of the plurality of depth indication bands having a width and being positioned at a different distance from the beveled tip,
a guide providing the distance from the beveled tip to each depth identification band,
wherein a width of one of the plurality of depth indication bands is greater than a width of a remainder of the plurality of depth indication bands,
wherein a width of the one of the plurality of depth indication bands nearest to the beveled tip is angled with respect to the first end of the collar tube thereby providing an indication of a proper angle of insertion of the needle shaft, and
wherein the first end of the collar tube contacts the adapter and the second end of the collar tube aligns with one of the plurality of depth indication bands.

12. The kit for administering medication and withdrawing biological fluids as recited claim 11, wherein each of the plurality of depth indication bands have various shades of the same color.

13. The kit for administering medication and withdrawing biological fluids as recited in claim 12, wherein each of the plurality of depth indication bands is a different shade of grey.

14. The kit for administering medication and withdrawing biological fluids as recited claim 11, wherein each of the plurality of depth indication bands is a different color.
